(19) United States
(12) Patent Application Publication
(10) Pub. No.: US 2026/0039147 A1
(43) Pub. Date: Feb. 5, 2026

(54) RECEIVE COIL ARRANGEMENTS FOR LEADLESS RECHARGEABLE EPICARDIAL PACEMAKER

(71) Applicant: Medtronic, Inc., Minneapolis, MN (US)

(72) Inventors: Rajesh V. Iyer, Eden Prairie, MN (US); Andrew J. Thom, Maple Grove, MN (US); Paul B. Young, New Richmond, WI (US); Teresa A. Whitman, Dayton, MN (US); Matthew D. Bonner, Plymouth, MN (US); Erik J. Hovland, Minnetonka, MN (US); Raymond W. Usher, Coon Rapids, MN (US); Andrew J. Ries, Lino Lakes, MN (US)

(21) Appl. No.: 18/997,664

(22) PCT Filed: Jul. 18, 2023
(86) PCT No.: PCT/IB2023/057338

§ 371 Date: Jan. 22, 2025

Related U.S. Application Data
(60) Provisional application No. 63/369,865, filed on Jul. 29, 2022.

(51) Int. Cl. H02J 50/12; A61N 1/375; A61N 1/378

(52) U.S. Cl. CPC H02J 50/12; A61N 1/3756; A61N 1/3787

(57) ABSTRACT

An implantable medical device (IMD) with electrodes near tissue of a patient and a rechargeable energy storage device. Power receiving circuitry receives electrical energy a wireless power transmitting device. The power receiving circuitry includes one or more secondary coils arranged to efficiently receive the wireless power when the primary coil is at any angle relative to the IMD. The IMD includes a non-conductive, hermetically sealed housing that encloses the device circuitry, including the rechargeable energy storage device, power receiving circuitry, processing circuitry, electrical stimulation circuitry and other components to perform the functions of the IMD.

This application claims the benefit of U.S. Provisional Patent Application Ser. No. 63/369,865, filed Jul. 29, 2022, the entire content of which is incorporated herein by reference.

### TECHNICAL FIELD

The disclosure relates wireless power transfer and more specifically, power transfer for medical devices.

### BACKGROUND

Implantable medical devices that deliver electrical stimulation therapy and monitor bioelectrical signals, and other signals, from a patient may include leads to place electrodes proximal to target tissue of a patient. In other examples, implantable medical devices may be leadless, and include electrodes on the housing of the implantable medical device to monitor the patient and/or deliver electrical stimulation therapy. Implantable medical devices may include an electrical energy storage device, such as a capacitor, rechargeable battery, or a non-rechargeable battery, e.g., a primary battery.

### SUMMARY

In general, the disclosure describes a leadless implantable medical device (IMD) that includes electrodes proximate to target tissue of the patient and a rechargeable electrical energy storage device. The IMD of this disclosure includes wireless power receiving circuitry configured to receive wireless electrical energy from a primary coil of a power transmitting device. The power receiving circuitry includes one or more secondary coils arranged to efficiently receive the wireless power from the primary coil when the primary coil is at any angle relative to the IMD. In some examples, such as an implantable epicardial device, the IMD may move frequently and randomly relative to the primary coil based on movement of the patient, e.g., as the heart beats.

In addition, the IMD of this disclosure includes a non-conductive, hermetically sealed housing that encloses the device circuitry, including the rechargeable energy storage device, power receiving circuitry, processing circuitry, sensing circuitry, electrical stimulation circuitry and other components to perform the functions of the IMD. The electrodes are mounted to or integral to the housing and the electrodes are arranged to be proximate to target tissue of the patient, e.g., cardiac tissue, nerve tissue, muscle tissue and other locations for the patient.

In one example, this disclosure describes an implantable medical device comprising circuitry configured to receive wireless power; an electrical energy storage device configured to provide electrical energy to the circuitry, wherein the circuitry is configured to charge the energy storage device using the wireless power; a secondary antenna configured to configured to receive the wireless power and conduct the wireless power to the circuitry, the secondary antenna comprising a first coil that defines a first aperture, the first aperture oriented in a first direction; a second coil that defines a second aperture, the second aperture oriented in a second direction substantially orthogonal to the first aperture; a third coil that defines a third aperture, wherein the third aperture: is oriented in a third direction substantially orthogonal to the first direction and the second direction; has at least twice the area as either of the first aperture and the second aperture.

In another example, this disclosure describes a wireless power transfer system comprising two or more electrodes configured to be placed proximal to target tissue of a patient; an implantable medical device comprising circuitry configured to: measure bioelectrical signals of the patient via the two or more electrodes; and receive wireless power; an electrical energy storage device configured to provide electrical energy to the circuitry, wherein the circuitry is configured to charge the energy storage device using the wireless power; a secondary antenna configured to configured to receive the wireless power and conduct the wireless power to the circuitry, the secondary antenna comprising a first coil that defines a first aperture, the first aperture oriented in a first direction; a second coil that defines a second aperture, the second aperture oriented in a second direction substantially orthogonal to the first aperture; a third coil that defines a third aperture, wherein the third aperture: is oriented in a third direction substantially orthogonal to the first direction and the second direction; has at least twice the area as either of the first aperture and the second aperture.

In another example, this disclosure describes a method of manufacturing a wireless power receiving device comprising forming a first coil around a ferrite core, wherein the first coil defines a first aperture, the first aperture oriented in a first direction; forming a second coil around the ferrite core, wherein the second coil defines a second aperture, the second aperture oriented in a second direction substantially orthogonal to the first aperture; arranging the ferrite core proximal to circuitry and electrically connecting the first coil and the second coil to the circuitry; arranging an electrical energy storage device proximal to the circuitry and electrically connecting the electrical energy storage device to the circuitry, wherein the circuitry is configured to receive wireless power via the first coil and the second coil, wherein the circuitry is configured to charge the energy storage device using the wireless power received during a charging session, wherein the electrical energy storage device is configured to provide electrical energy to the circuitry; and forming a third coil that defines a third aperture, wherein the third aperture: is oriented in a third direction substantially orthogonal to the first direction and the second direction; has at least twice the area as either of the first aperture and the second aperture.

In another example, this disclosure describes a method of manufacturing a wireless power receiving device comprising assembling a receiving coil of a plurality of receiving coils, wherein each receiving coil of the plurality of receiving coils comprises one or more coil windings comprising a conductive material configured to carry electrical current; measuring the inductance of each receiving coil; calculating values for respective tuning circuitry associated with each receiving coil based on an operating frequency range for the respective receiving coil; and verifying a resonance frequency for each receiving coil circuit, wherein each receiving coil circuit comprises the respective receiving coil and respective tuning circuitry.

In one example, this disclosure describes an implantable medical device comprising two or more electrodes configured to be placed proximal to target tissue of a patient; circuitry; and a non-conductive, hermetically scaled housing: configured to enclose the circuitry, wherein the circuitry is configured to measure bioelectrical signals of the patient via the two or more electrodes; comprising a conductive ferrule configured to: hermetically seal the housing; and act as a first electrode of the two or more electrodes.

In another example, this disclosure describes a wireless power transfer system includes one or more antennae configured to receive wireless power from a power transmitting device; an implantable medical device (IMD) includes two or more electrodes configured to be placed proximal to target tissue of a patient; circuitry; and a non-conductive, hermetically sealed housing: configured to enclose the circuitry, wherein the circuitry is configured to measure bioelectrical signals of the patient via the two or more electrodes; comprising a conductive ferrule configured to: hermetically seal the housing; act as a first electrode of the two or more electrodes.

In another example, this disclosure describes a method of manufacturing a wireless power receiving device comprising assembling a cover to a base, wherein: a first circumference of the cover aligns with second circumference of the base; the circumference of the cover comprises a conductive weld ring; the circumference of the cover comprises a non-conductive gap in the weld ring; bonding the cover to the base; and hermetically sealing the cover to the base, where hermetically sealing the cover to the base comprises: bonding the non-conductive gap in the cover to the base with a non-conductive bond.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual drawing illustrating an example medical system in conjunction with a patient according to various examples described in this disclosure.

FIG. 2 is a functional block diagram illustrating an example configuration of an IMD of a medical system.

FIG. 3 is a conceptual diagram illustrating an example rechargeable IMD according to one or more techniques of this disclosure.

FIG. 4A is a conceptual diagram illustrating an example X-coil and Y-coil secondary antennae according to one or more techniques of this disclosure.

FIG. 4B is a conceptual diagram of a D-shaped Z-coil, according to one or more techniques of this disclosure.

FIGS. 5A and 5B are conceptual diagrams illustrating an electrical conductor configured to form a receive coil for an implantable medical device according to various examples described in this disclosure.

FIG. 6A is a conceptual diagram of a flat, spiral shaped Z-coil, according to one or more techniques of this disclosure.

FIG. 6B is a conceptual diagram of a flat, folded infinity wound coil configured as a Z-coil according to one or more techniques of this disclosure.

FIGS. 7A and 7B are conceptual diagrams illustrating an example spiral-wound coil implemented as an X-coil, according to one or more techniques of this disclosure.

FIG. 8 is a conceptual diagram illustrating an examples of receive coils implemented as a Y-coil according to one or more techniques of this disclosure.

FIG. 9 is a conceptual diagram illustrating an example planar coil implemented to receive wireless energy in multiple planes, according to one or more techniques of this disclosure.

FIG. 10 is a schematic diagram illustrating and example three coil wireless power receiving circuit, according to one or more techniques of this disclosure.

FIGS. 11, 12 and 13 are flow charts illustrating example methods of manufacturing an implantable medical device according to one or more techniques of this disclosure.

### DETAILED DESCRIPTION

The disclosure describes implantable medical devices, including receive coil configurations for implantable medical devices and associated techniques, structures, and assemblies configured to provide recharging of power sources located within medical devices that have been implanted within a patient. An implantable medical device (IMD) may include a receive coil (also referred to as a secondary coil) positioned within a portion of the housing of the device. The receive coil may be coupled to recharging circuitry and configured so that currents induced in the receive coil provide a recharging current for rechargeable power source of the IMD. The receive coil may be made from one or more windings formed from individual electrical conductors, such as multi-strand wires.

In some examples, the receive coil may have a curved shape corresponding to an inner surface of the housing of the IMD, and in some examples, the coil may be proximal to a flexible ferrite sheet. In other examples, the receive coil of this disclosure may also include coils wound about a ferrite of various shapes, e.g., cylindrical, rectangular and similar shapes. In some examples, externally generated magnetic field(s) that are imposed onto the receive coil, may be enhanced by the presence of the ferrite near to the secondary coil, for the purpose of providing inductive recharging of a power source located with the IMD, such as a battery or a supercapacitor.

The housing of a power receiving device, such as a rechargeable IMD, may impact the amount of energy received by the power receiving device. A metallic housing for a power receiving device may block radio-frequency (RF) transmissions and may also result in eddy currents in the conductive housing. The conductive housing may also absorb the transmitted RF energy and in in some examples, may raise the temperature of the surrounding patient tissue, which may require reducing the amount of energy transmitted. In contrast, the non-conductive, hermetically sealed housing for the power receiving devices of this disclosure may be RF transparent, which may provide the advantage of improved energy transfer efficiency, compared to other types of systems. Improved power transfer efficiency may provide benefits even for power receiving devices implanted deeper in the patient tissue than subcutaneous implants under the skin.

When there is a need to recharge a power source of an IMD that includes a receive coil configuration and housing as described in this disclosure, a power transmitting device may generate a magnetic field (or a resultant magnetic field formed by a plurality of magnetic fields) using one or more transmit coils (also referred to as primary coils). The resultant magnetic field(s) imposed on the device may induce electrical current(s) into one or more of the windings of the receive coil. The induced electrical current or currents may be used to recharge the power source of the IMD and/or to provide the electrical power used to directly operate the device.

In the example of IMDs used to monitor or treat cardiac symptoms of a patient, the IMD may sense cardiac electrograms (EGMs) and/or other physiological signals or characteristics of a patient. In some examples, electrodes used by IMDs to sense cardiac EGMs are integrated with a housing of the IMD and/or coupled to the IMD via one or more elongated leads. Such IMDs may facilitate relatively longer-term monitoring of patients during normal daily activities and may periodically transmit collected data to a network service, such as the Medtronic Carelink™ Network.

In some examples of IMDs, may operate using a primary (non-rechargeable) battery with a finite energy reservoir. Once a primary battery is exhausted, replacement of the device may be required, and although replacement of the device may be minimally invasive, it may still present procedural risks for the patient. In addition, limits on the available battery energy may result in limits to therapy and/or monitoring features available to the patient.

The ability to recharge the power source of an IMD, for example within a one-hour recharging period of time on a monthly or yearly cycle, without the need to explant the device to do so, may result in at least some benefits, including use of a smaller power source to help miniaturize the IMD itself, and to allow more power, and thus greater functionality for the implanted medical device by providing an overall longer mission lifespan for the device using a smaller-sized power source.

Throughout the disclosure, a reference to a “receive coil” or “secondary coil” refers to a coil winding formed from an electrical conductor that may or may not be coupled with one or more additional coil windings to form a receive coil for an implantable medical device. The use of the term “antenna” may be used in place of or interchangeably with the term “coil” in any context referring to a coil winding that is coupled to recharging circuitry of an implantable medical device and that may be configured to have current induced into the coil winding for the purpose of providing electrical energy to the implantable medical device. In this disclosure, a secondary coil may include multiple receive coil elements and arrangements in which each of the coils may vary with respect to aperture area, orientation, number of turns, wire type (e.g., Litz or magnet wire) or composition (copper, silver, gold, etc.), and proximity (or not) to ferrite core or ferrite sheet.

Throughout the disclosure reference to a “magnetic field” or to “magnetic fields” in the context of a magnetic field or magnetic fields generated by a transmit coil or coils (also called a primary coil) external to an IMD. In general, such a magnetic field or magnetic fields have parameter (e.g., amplitude or phase) that varies in time, or that varies in time with respect to the magnetic field direction of the magnetic field, resulting in a time rate of change of the net magnetic flux intensity imposed onto the coil windings of the receive coil, and a corresponding change in the electro-motive force (emf) configured to generate a current or currents in the one or more coil windings.

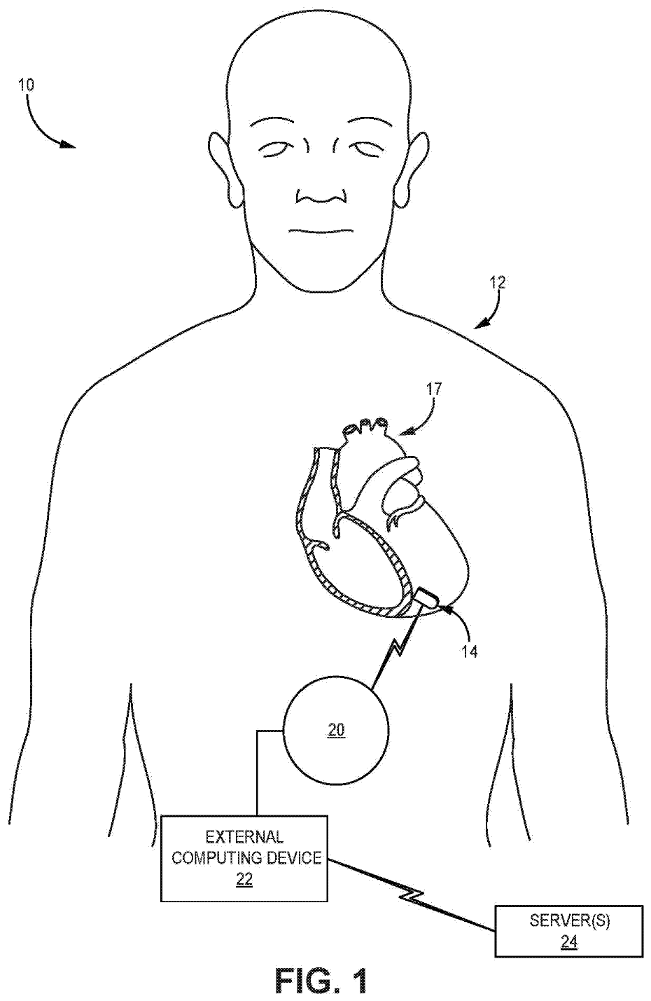

FIG. 1 is a conceptual drawing illustrating an example medical system 10 in conjunction with a patient 12 according to various examples described in this disclosure. The systems, devices, and methods described in this disclosure may include example configurations of a receive coil (not shown in FIG. 1) located within an IMD 14, for charging of IMD 14, as illustrated and described with respect to FIG. 1. For purposes of this description, knowledge of cardiovascular anatomy and functionality is presumed, and details are omitted except to the extent necessary or desirable to explain the context of the techniques of this disclosure. System 10 includes rechargeable IMD 14, implanted at or near the site of a heart 18 of a patient 12; a transmit coil 20 coupled to external computing device 22; and one or more servers 24. The systems, devices, and methods described herein may provide efficient inductive coupling of an external computing device 22 to the electrical circuitry that is internal to IMD 14. Though described in terms of a medical device system including IMD 14, in other examples, the wireless power transfer techniques of this disclosure may apply to other types of devices. Examples of other types of devices may include mobile communication devices, sensor devices, actuator devices, or any other device in which receiving wireless power may be useful.

IMD 14 may be in wireless communication with at least one of external computing device 22, servers 24, and other devices not pictured in FIG. 1. In some examples, IMD 14 may implanted outside of a thoracic cavity of patient 12 (e.g., subcutaneously in the pectoral location illustrated in FIG. 1). In other examples, IMD 14 may be positioned near the sternum near or just below the level of the heart of patient 12. e.g., at least partially within the cardiac silhouette. In other examples, IMD 14 may be implanted proximate to, attached to, or on the epicardium of heart 17, as shown in FIG. 1. In other examples, IMD 14 may be located in other locations on patient 12, including for monitoring and stimulation of the tibial nerve, sacral nerve, spinal cord, vagal nerve, deep brain stimulation, located at or near one or more organs or other locations.

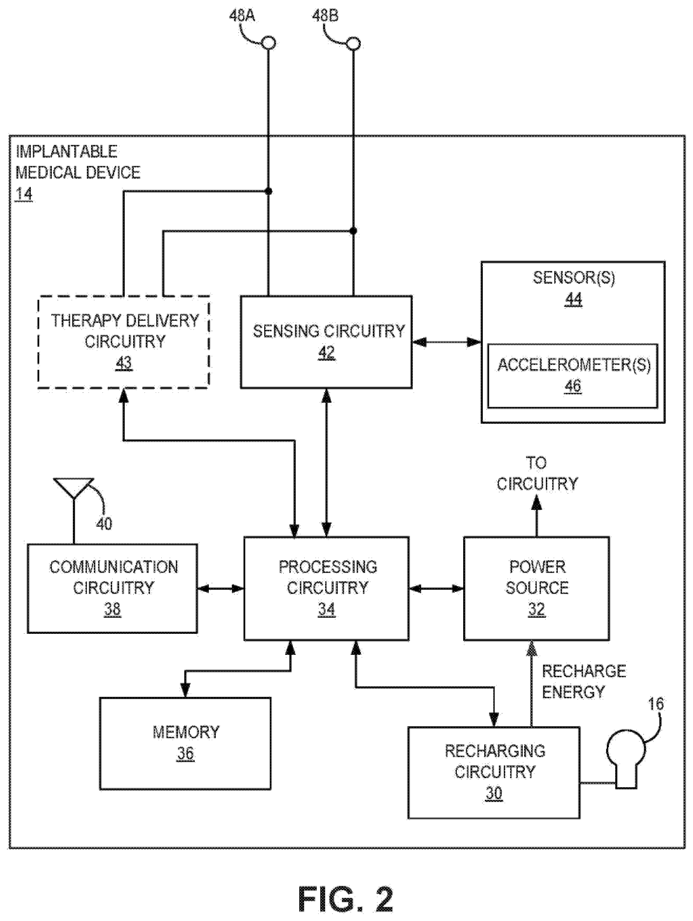

IMD 14 includes a plurality of electrodes 48 (FIG. 2) and may be configured to sense a cardiac electrogram (EGM) and other bioelectrical signals via the plurality of electrodes. In some examples, electrodes may be integrated with the non-conductive, RF transparent housing of IMD 14. In various examples, IMD 14 may represent a cardiac monitor, a defibrillator, a cardiac resynchronization pacer/defibrillator, a pacemaker, an implantable pressure sensor, a neurostimulator, glucose monitor, drug pump, pulse wave velocity measurement device or any other implantable or external medical device.

In some examples, external computing device 22 may be a computing device with a display viewable by the user and an interface for providing input to external computing device 22 (i.e., a user input mechanism). In some examples, external computing device 22 may be a notebook computer, tablet computer, workstation, one or more servers, cellular phone, personal digital assistant, or another computing device that may run an application that enables the computing device to interact with IMD 14. External computing device 22 is configured to communicate with IMD 14 and, optionally, other device (not illustrated in FIG. 1), and one or more servers 24, e.g., via wireless communication. External computing device 22, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies).

External computing device 22 may be used to configure operational parameters for IMD 14. External computing device 22 may be used to retrieve data from IMD 14. The retrieved data may include values of physiological parameters measured by IMD 14, indications of episodes of arrhythmia or other maladies detected by IMD 14, and physiological signals recorded by IMD 14. For example, external computing device 22 may retrieve cardiac EGM segments recorded by IMD 14, e.g., due to IMD 14 determining that an episode of arrhythmia or another malady occurred during the segment, or in response to a request to record the segment from patient 12 or another user. In some examples, one or more remote computing devices may interact with IMD 14 in a manner similar to external computing device 22, e.g., to program IMD 14 and/or retrieve data from IMD 14, via a network.

In some examples, external computing device 22 may be referred to as a wireless power transmitting device or recharger. External computing device 22 may output and control the wireless power delivery to IMD 14. In other examples system 10 may include two separate external computing devices, one for controlling the wireless power delivery (as shown) and a separate computing device may program and update functional parameters of IMD 14 (not shown in FIG. 1).

In some examples, primary coil 20 may be implemented as one or more coils separate from external computing device 22, such as on a paddle or similar device. In other examples, primary coil 20 may be embedded in furniture, or in a pad attached to furniture. In some examples primary coil 20 may be within a mattress, a chair, an automobile seat or similar locations such that patient 12 may conveniently deliver wireless power to IMD 14.

In various examples, IMD 14 may include one or more additional sensor circuits configured to sense a particular physiological or neurological parameter associated with patient 12. For example, IMD 14 may include a sensor operable to sense a body temperature of patient 12 in a location of the IMD 14, or at the location of the patient where a temperature sensor coupled by a lead to IMD 14 is located (not shown in FIG. 1). In another example, IMD 14 may include a sensor configured to sense motion or position, e.g., and accelerometer, to sense steps taken by patient 12 and/or a position or a change of posture of patient 12. In various examples, IMD 14 may include a sensor that is configured to detect breaths taken by patient 12. In various examples, IMD 14 may include a sensor configured to detect heartbeats of patient 12. In various examples, IMD 14 may include a sensor that is configured to measure systemic blood pressure of patient 12 or other biological measurements.

In some examples, system 10 may include one or more other sensors (not shown in FIG. 1) implanted within patient 12, that is, implanted below at least the skin level of the patient. In some examples, one or more of the sensors of IMD 14 may be located externally to patient 12, for example as part of a cuff or as a wearable device, such as a device imbedded in clothing that is worn by patient 12. In various examples, IMD 14 may be configured to sense one or more physiological parameters associated with patient 12, and to transmit data corresponding to the sensed physiological parameter or parameters to external computing device 22.

Transmission of data from IMD 14 to external computing device 22 in various examples may be performed via wireless transmission, using for example any of the formats for wireless communication described above. In various examples, IMD 14 may communicate wirelessly to an external device (e.g., an instrument or instruments) other than or in addition to external computing device 22, such as a transceiver or an access point that provides a wireless communication link between IMD 14 and a network. In various examples, a transceiver is communication circuitry included within recharging circuitry 30, wherein communication circuitry of external computing device 22 is configured to communicate with IMD 14 during the recharging process, as further described below. Examples of communication techniques used by any of the devices described above with respect to FIG. 1 may include radiofrequency (RF) telemetry, which may be an RF link established via Bluetooth®, Wi-Fi, or medical implant communication service (MICS).

In some examples, system 10 may include more or fewer components than depicted in FIG. 1. For example, in some examples, system 10 may include multiple additional IMDs, such as implantable pacemaker devices or other IMDs, implanted within patient 12. In these examples, rechargeable IMD 14 may function as a hub device for the other IMDs. For example, the additional IMDs may be configured to communicate with the rechargeable IMD 14, which would then communicate to the external computing device 22, such as a user's smartphone, via a low-energy telemetry protocol.

Rechargeable IMD 14 may provide a theoretically infinite energy capacity, in that IMD 14 may not need to be replaced or otherwise removed. Accordingly, IMD 14 may provide the ability to more frequently telemeter information, as well as more active titration of therapies.

For the remainder of the disclosure, a general reference to a medical device system may refer collectively to include any examples of medical device system 10, a general reference to IMD 14 may refer collectively to include any examples of IMD 14, a general reference to sensor circuits may refer collectively to include any examples of sensor circuits of IMD 14, and a general reference to an external device may refer collectively to any examples of external computing device 22.

FIG. 2 is a functional block diagram illustrating an example configuration of IMD 14 of medical system 10 of FIG. 1. In the illustrated example, IMD 14 includes receive coil 16, recharging circuitry 30, rechargeable power source 32, processing circuitry 34, memory 36, communication circuitry 38, communication antenna 40, sensing circuitry 42, sensor(s) 44 including accelerometer(s) 46, and electrodes 48A and 48B (collectively, “electrodes 48”). Although the illustrated example includes two electrodes 48, in other examples IMD 14 may be coupled to more than two electrodes 48.

Processing circuitry 34 may include fixed function circuitry and/or programmable processing circuitry. Processing circuitry 34 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 34 may include multiple components, such as any combination of one or more microprocessors, one or more controllers, one or more DSPs, one or more ASICs, or one or more FPGAs, as well as other discrete or integrated logic circuitry. The functions attributed to processing circuitry 34 herein may be embodied as software, firmware, hardware or any combination thereof.

Sensing circuitry 42 is coupled to electrodes 48. Sensing circuitry 42 may sense signals from electrodes 48, e.g., to produce a cardiac EGM, to facilitate monitoring the electrical activity of the heart. Processing circuitry 34 may receive indications from sensing circuitry 42 to determine heart rates or heart rate variability, or to detect arrhythmias (e.g., tachyarrhythmias or bradycardia), patient breathing rhythm, biological impedance or other bioelectrical signals via electrodes 48. Sensing circuitry 42 also may monitor signals from sensors 44, which may include one or more accelerometers 46, pressure sensors, temperature sensors and/or optical sensors, as examples. In some examples, sensing circuitry 42 may include one or more filters and amplifiers for filtering and amplifying signals received from electrodes 48 and/or sensors 44.

Sensing circuitry 42 and/or processing circuitry 34 may be configured to detect cardiac depolarizations (e.g., P-waves of atrial depolarizations or R-waves of ventricular depolarizations) when the cardiac EGM amplitude crosses a sensing threshold. For cardiac depolarization detection, sensing circuitry 42 may include a rectifier, filter, amplifier, comparator, and/or analog-to-digital converter, in some examples. In some examples, sensing circuitry 42 may output an indication to processing circuitry 34 in response to sensing of a cardiac depolarization. In this manner, processing circuitry 34 may receive detected cardiac depolarization indicators corresponding to the occurrence of detected R-waves and P-waves in the respective chambers of heart. Processing circuitry 34 may use the indications of detected R-waves and P-waves for determining inter-depolarization intervals, heart rate, and detecting arrhythmias, such as tachyarrhythmias, bradyarrhythmias, and asystole.

Sensing circuitry 42 may also provide one or more digitized cardiac EGM signals to processing circuitry 34 for analysis, e.g., for use in cardiac rhythm discrimination. In some examples, processing circuitry 34 may store the digitized cardiac EGM in memory 36. Processing circuitry 34 of IMD 14, and/or processing circuitry of another device that retrieves data from IMD 14, may analyze the cardiac EGM.

In some examples, IMD 14 may include therapy delivery circuitry 43. Therapy delivery circuitry 43 may be configured to output electrical stimulation therapy to target tissue of the patient, such as to cardiac tissue, nerve tissue and similar patient tissue. In some examples, processing circuitry 34 may control one or more parameters of electrical stimulation from therapy delivery circuitry 43 based on bioelectrical signals sensed by sensing circuitry 42. For example, processing circuitry 34 may determine that ventricular contraction is later than expected, e.g., a duration since a previous contraction exceeds a duration threshold. Processing circuitry may cause therapy deliver circuitry to output electrical stimulation therapy in the form of a pacing pulse to cause the heart of the patient to contract.

Communication circuitry 38 may include any suitable hardware, firmware, software or any combination thereof for communicating with another device, such as external computing device 22, another networked computing device, or another IMD or sensor. Under the control of processing circuitry 34, communication circuitry 38 may receive downlink telemetry from, as well as send uplink telemetry to external computing device 22 or another device with the aid of an internal or external antenna, e.g., antenna 40. In addition, processing circuitry 34 may communicate with a networked computing device via an external device (e.g., external computing device 22 of FIG. 1) and a computer network, such as the Medtronic CareLink® Network. Antenna 40 and communication circuitry 38 may be configured to transmit and/or receive signals via inductive coupling, electromagnetic coupling, Near Field Communication (NFC), Radio Frequency (RF) communication, Bluetooth, Wi-Fi, or other proprietary or non-proprietary wireless communication schemes. Communication antenna 40 may telemeter data at a high frequency, such as around 2.4 gigahertz (GHz). IMD 14 may receive messages from external computing device 20, another medical device worn, or implanted in, patient 12 or some other source, which may cause IMD 14 to take a measurement via the electrodes, or other sensors, or to deliver electrical stimulation therapy.

In some examples, memory 36 includes computer-readable instructions that, when executed by processing circuitry 34, cause IMD 14 and processing circuitry 34 to perform various functions attributed to IMD 14 and processing circuitry 34 herein. Memory 36 may include any volatile, non-volatile, magnetic, optical, or electrical media, such as a random-access memory (RAM), read-only memory (ROM), non-volatile RAM (NVRAM), electrically erasable programmable ROM (EEPROM), flash memory, or any other digital media. Memory 36 may store, as examples, programmed values for one or more operational parameters of IMD 14 and/or data collected by IMD 14, e.g., posture, heart rate, activity level, respiration rate, therapy delivery statistics, and other parameters, as well as digitized versions of physiological signals sensed by IMD 14, for transmission to another device using communication circuitry 38.

IMD 14 includes a rechargeable power source 32 that may be coupled to the electronic circuitry provided in IMD 14 and is configured to provide electrical power to these circuits outside of a charging session, e.g., when not receiving wireless power from a primary coil. Power source 32 may be an electrical energy storage device that is inductively rechargeable by imposing one or more magnetic fields onto IMD 14, wherein energy from these imposed field(s) may induce an electrical energy into receive coil 16 and, thereby, to recharging circuitry 30.

As shown in FIG. 2, device recharging circuitry 30 is coupled to power source 32 and may receive electrical energy induced in receive coil 16 by one or more electromagnetic fields imposed on the coil during a charging session, and to regulate the energy to provide a level of energy that is provided to power source 32 for the purpose of recharging power source 32 and/or powering the other circuitry included as part of IMD 14. Device recharging circuitry 30 may perform various energy conditioning functions to the energy inductively generated in receive coil 16 during the charging session by the primary coil, e.g., primary coil 20 described above in relation to FIG. 1. For example recharging circuitry 30 may provide rectification, voltage level regulation, current level regulation, and/or other signal processing functions to generate the “recharging energy” provided to charge power source 32.

In the illustrated example, IMD 14 includes processing circuitry 34 and an associated memory 36, sensing circuitry 42, one or more sensors 44, and the communication circuitry 38 coupled to antenna 40 as described above. However, IMD 14 need not include all of these components, or may include additional components.

Processing circuitry 34 may be configured to provide information including a state of charge, and/or temperature information related to a battery, e.g., a battery located in IMD 14, determining a level of inductive coupling, e.g., energy level being generated in a receive coil located in IMD 14 as a result of an electromagnetic field or fields being imposed on IMD 14, and generate information related to this inductively received energy for transmission by the communication antenna or separate antenna and associated power conditioning circuitry of IMD 14.

In various examples, processing circuitry 34 is coupled to device recharging circuitry 30, and receives information, such as a level of current, that is being induced in coil 16 as a result of electrical energy received by the antenna via magnetic energy imposed on IMD 14 for the purpose of recharging power source 32. Processing circuitry 34 may provide this and other information, for example charge rate and temperature information associated with the power source 32, in the form of an output signal to communication circuitry 38 for transmission from IMD 14 to one or more external devices, such as external computing device 22 (FIG. 1). This transmitted information may be used by the external device(s) to control one or more aspects of the recharging process.

For example, positioning of and/or a level of power being applied to a recharging coil or a pair of coils located externally to IMD 14 and generating the magnetic field or fields being imposed on IMD 14 may be controlled using this information transmitted from IMD 14. External computing device 22, described above in relation to FIG. 1, may set electrical parameters used to energize and control the primary coil generating the magnetic field or fields imposed onto IMD 14 for the purpose of recharging the power source 32 based on information transmitted from IMD 14. In addition, processing circuitry of external computing device 22 may use other information such as temperature and field intensity information transmitted from IMD 14, may be used to control the recharging process, for example by regulating the field strength being generated by the external coil(s), or for example to shut off the external coil(s) to stop the recharging process.

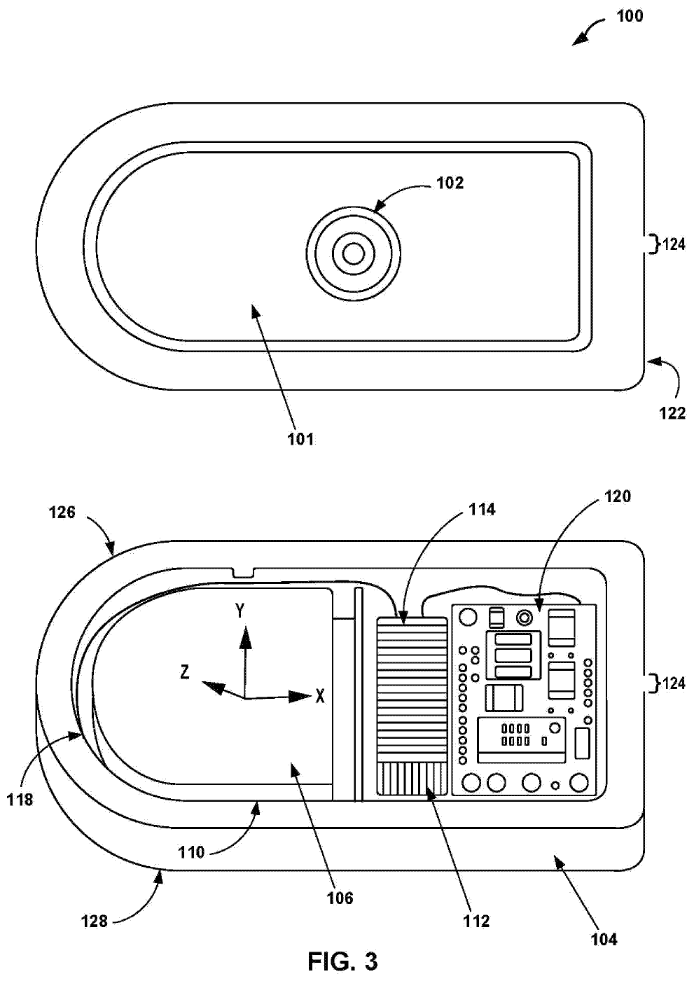

FIG. 3 is a conceptual diagram illustrating an example rechargeable IMD according to one or more techniques of this disclosure. IMD 100 is an example of IMD 14 described above in relation to FIGS. 1 and 2. IMD 100 may include an RF transparent cover 101, and RF transparent base 104 that contains one or more power receiving antennae, circuitry 120, and an electrical energy storage device 106. In some examples, base 104 includes a bottom and sides along with cover 101 provides a hermetically sealed housing enclosing the circuitry and other components of IMD 100. In other examples, base 104 is implemented as side walls and cover 101, along with a second RF transparent cover 128, provides the hermetically sealed housing. Cover 101 and cover 128 may be made from a variety of materials including ceramics such as include polycrystalline alumina, single crystal alumina (sapphire), zirconia, zirconia toughened alumina, alumina toughened zirconia, glass, and other similar RF transparent materials.

Circuitry 120 may include the processing circuitry, communication circuitry, sensing circuitry, stimulation therapy circuitry and other components described above for IMD 14 in relation to FIG. 2. Circuitry 120 may connect to a telemetry antenna (not shown in FIG. 3), e.g., communication antenna 40 of FIG. 2, which may be located below circuitry 120. Circuitry 120 may connect to electrode 102, and to a conductive weld ring 122, e.g., a conductive ferrule, which acts as a second electrode. Weld ring 122 is a conductive material, such as a metallic ring, tantalum, titanium, niobium, or other conductive material, that may seal cover 101 to base 104 using any of a variety of processes, including laser welding, temperature diffusion bonding or similar scaling processes. In examples in which IMD 100 includes a second cover, IMD 100 may also include a second weld ring, used to seal second cover 128 to base 104. The second weld ring may connect to circuitry 120 as a third electrode. The electrodes may provide a path for bioelectrical sensing and electrical stimulation therapy delivery, as described above for electrodes 48 in relation to FIGS. 1 and 2. In some examples second cover 128 may include another electrode, similar to electrode 102 (not shown in FIG. 3) that also connects to circuitry 120.

In some examples, a weld ring 126 that is bonded to the case, e.g., bonded to base 104, and the weld ring that is bonded to the cover, e.g., weld ring 122 make an electrical connection during manufacturing. For cover 101, weld ring 122 mates with weld ring 126 as cover 101 closes over the case and are laser welded, or otherwise bonded to make the hermetic device enclosure. Effectively, at that point, weld ring 122 and weld ring 126 become a single electrode. When implanted in patient tissue, electrode formed by weld ring 122 and 126 may act as the return electrode (anode) for the IMD. Electrode 102 may act as the stim electrode (cathode). Similarly, second cover 128 may include a weld ring (not shown in FIG. 3) that bonds to a weld ring on base 104 and also acts as an electrode proximal to target tissue of the patient. Electrode 102 may be electrical isolated on cover 101 from weld ring 122, e.g., before and after assembly and connection to circuitry 120.

Electrical energy storage device 106 may be a battery, a supercapacitor or similar energy storage device. Electrical energy storage device 106 may provide electrical power for circuitry 120 to perform the sensing and other functions of IMD 100. Circuitry 120 may include recharging circuitry configured to conduct wireless power received by the power receiving antennae to electrical energy storage device 106, which may have the same or similar functions to recharging circuitry 30 described above in relation to FIG. 2.

In some examples, weld rings 122 and 126 may bond to the complete circumference of base 104 and cover 101. In other examples, weld ring 122, and weld ring 126, may also include a non-conductive gap 124. The non-conductive gap 124 may ensure that weld ring 122 is an incomplete conductive ring, which may avoid eddy currents in weld ring 122 caused by the electromagnetic field generated by the primary coil, e.g., primary coil 20 of FIG. 1. In some examples, non-conductive gap 124 may be filled with a biocompatible non-conductive material after bonding the cover to base 104. For example, weld ring 122 and weld ring 126 may be bonded using a laser weld process, and gap 124 filled after the laser weld process, e.g., using a low temperature bonding process. In other examples, gap 124 may filled before or filled during the same bonding process as for weld ring 122 and 126, e.g., with a low temperature bonding process. In other examples, the weld rings of this disclosure may include the entire circumference of cover 101 and base 104 and the weld ring may have no gap. In some examples the low temperature bonding process may include a diffusion bond seal, such as niobium (Nb) sputter, to bond the weld rings at the interface of cover 101 and base 104.

The power receiving antennae of IMD 100 may include Y-coil 114, X-coil 112 and Z-coil 118, which are examples of receive coil 16 described above in relation to FIG. 2. Y-coil 114. X-coil 112 and Z-coil 118 may act as secondary coil, e.g., secondary antennae, to receive wireless power from a primary coil, e.g., transmit coil 20 described above in relation to FIG. 1. The three-axis orientation of the secondary antennae of IMD 100 may provide efficient wireless power transfer without regard for the relative orientation of primary coil 20 and IMD 100. Because IMD 100 may be located proximal to the epicardium of the heart of a patient, IMD 100 may be moving almost constantly because of the undulations of the heart during the cardiac cycle.

In some examples, X-coil 112 and Y-coil 114 may be wrapped around a ferrite core (not visible in FIG. 3). A rectangular shaped ferrite core may result in the rectangular shape of X-coil 112 and Y-coil 114 as shown in FIG. 3. As shown in FIG. 3, the antenna aperture for X-coil 112 may be oriented in the X-direction and the antenna aperture for Y-coil 114 may be oriented in the Y-direction, e.g., orthogonal to X-coil 112. The ferrite core may provide improved magnetic coupling between the primary coil and X-coil 112 and Y-coil 114, when compared to secondary coils without a ferrite. In addition, the ferrite for X-coil 112 and Y-coil 114 may also provide improved magnetic coupling between the primary coil and Z-coil 118, as well as between the telemetry coil the communication antenna on an external computing device (not shown in FIG. 3).

In some examples, Z-coil 118 may be placed as shown in FIG. 3 around the perimeter of base 104, e.g., enclosing circuitry 120 and electrical energy storage device 106. IMD 100 may also include a flexible ferrite 110, placed next to, and conforming to the shape of Z-coil 118. In other examples (not shown in FIG. 3), Z-coil 118 may be implemented as a flat, spiral wound coil placed either beneath or above circuitry 120, X-coil 112 and Y-coil 114 and electrical energy storage device 106, e.g., parallel to the plane of cover 101. The flat coil example of Z-coil 118 may also have a flat ferrite sheet place parallel to the coil and cover 101 to improve the magnetic coupling. The aperture for Z-coil 118 is oriented in the Z-direction as shown for either the D-shaped or flat, spiral wound example, which is substantially orthogonal the aperture for X-coil 112 and Y-coil 114. In this disclosure, “substantially” or “approximately,” e.g., “substantially orthogonal” means within manufacturing and measurement tolerances. In other words, values that are approximately equal, are equal within the tolerances, and substantially orthogonal is orthogonal, within the tolerances.

Circuitry 120 may include tuning circuitry, such as tuning capacitors, for each receive coil, which may set the resonant frequency for each receive coil to be compatible with the wireless power transmitting device, described above in relation to FIG. 1. The aperture size, number of windings, and other characteristics of each receive coil may be different from one another and therefore the tuning circuitry may be different, e.g., different values for one or more tuning capacitors. For example, the larger antenna aperture of Z-coil 118 may provide improved wireless power reception, when compared to X-coil 112 and Y-coil 114, with the smaller aperture. Therefore, the tuning circuitry for Z-coil 118 may be different from the tuning circuitry for X-coil 112 and Y-coil 114 to ensure that all three receive coils operate with compatible resonance. In some examples all the receive coils may simultaneously conduct wireless energy to circuitry 120. The magnitude of conducted energy, e.g., the magnitude of current, may be different for each coil at any point in time and based on how a particular coil is oriented relative to the primary coil.

In some examples, the capacitance of a tuning capacitor for tuning circuitry may be determined based on the measured inductance and selected operating frequency or operating frequency range. Because of the different shape and different number of turns in each coil, the inductance for each coil (Ls) may be different for each coil. As one possible example, calculate capacitance for tuning circuitry based on

$$C = \frac{1}{( 4 p {i}^{2} * f r e {q}^{2} * L s )} .$$

where ‘freq’ is the operating recharge frequency, which may be within a range of frequencies, such as a frequency within 100 kHz-10 MHz. The operating recharge frequency may be the selected resonance frequency for the primary coil, e.g., primary coil 20 of FIG. 1, as well as an average resonance frequency for the receive coils. In some examples the operating frequency may be selected as frequency that efficiently transfers electrical energy between the primary and secondary coils, and a frequency that may be less likely to be absorbed by the tissue of the patient.

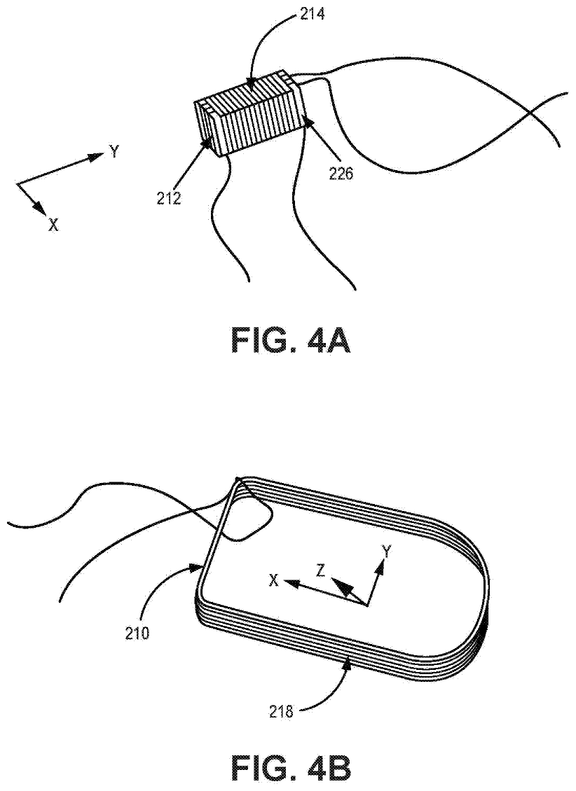

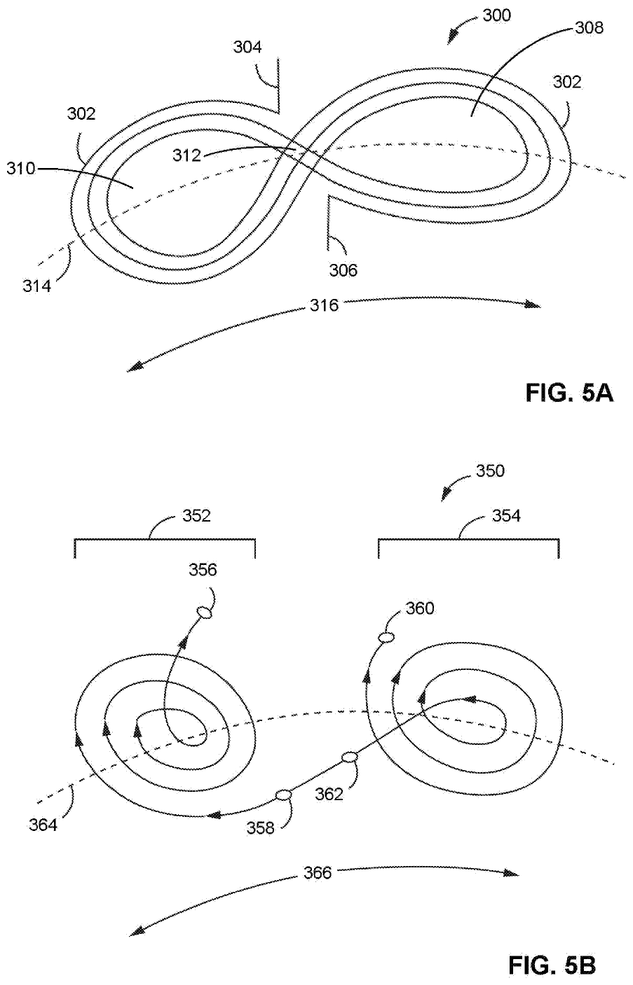

FIG. 4A is a conceptual diagram illustrating an example X-coil and Y-coil secondary antennae according to one or more techniques of this disclosure. X-coil 212 and Y-coil 214 are examples of X-coil 112 and Y-coil 114 described above in relation to FIG. 3. As noted above, X-coil 212 and Y-coil 214 may be wrapped around ferrite 226, which may provide improved magnetic coupling for the wireless power transmitted by a primary coil.

FIG. 4B is a conceptual diagram of a D-shaped Z-coil, according to one or more techniques of this disclosure. Z-coil 218 is an example of Z-coil 118 described above in relation to FIG. 3 and may be located around the perimeter of base 104, e.g., enclosing circuitry 120 and electrical energy storage device 106. In some examples, Z-coil 218 may also have a flexible ferrite sheet 210 proximal to Z-coil 218, as described above in relation to FIG. 3. The flexible ferrite sheet may be located either around the outside perimeter or inside perimeter, e.g., the periphery, of Z-coil 218.

In the example of FIG. 4B, Z-coil 218 is a D-shaped coil. However, in other examples, Z-coil 218 may be oval, circular, rectangular or any other shape. The shape of Z-coil 218 may depend on the space available inside the housing of the IMD. The aperture of Z-coil 218 may be larger than the aperture of X-coil 212 and Y-coil 214 described above in relation to FIGS. 3 and 4A. In some examples, the area of the aperture for Z-coil 218 may be at least double the area of the apertures for X-coil 212 and Y-coil 214.

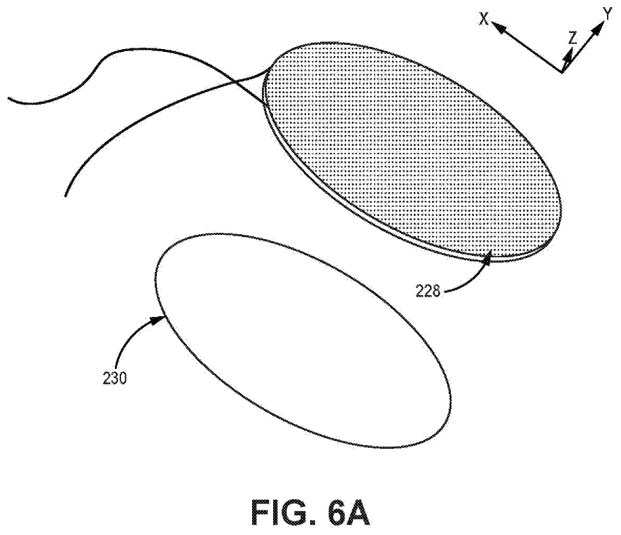

FIGS. 5A and 5B are conceptual diagrams illustrating an electrical conductor configured to form a receive coil for an implantable medical device according to various examples described in this disclosure. In the example of FIG. 5A, electrical conductor 302 is arranged to form a receive coil 300, which may be used for a device configured to receive wireless power, such as for an implantable medical device according to various examples described in this disclosure. In the example of FIG. 5A, a first end of electrical conductor 302 is electrically coupled to a first lead 304 and a second end of electrical conductor 302 is electrically coupled to a second lead 306. First lead 304 and second lead 306 may be configured to extend to and electrically couple receive coil 300 with wireless power receiving circuitry, such as recharging circuitry of an implantable medical device as described above in relation to FIGS. 1-4B. As described above, currents may be induced into receive coil 300 by magnetic field(s) imposed onto receive coil 300, e.g., by primary coil 20 connected to a wireless power transmitting device, depicted in FIG. 1. The received current may be used to recharge a power source of an implanted medical device coupled to the receive coil, and/or to directly power the operation of the electrical circuitry of the device.

In some examples, the overall thickness dimension of the receive coil 300 (e.g., a thickness dimension of receive coil 300) may be the thickness of the diameter of the electrical conductor 302. In other words, the coil winding of receive coil 300 as shown in FIG. 5A may be configured as a planar coil having any shape including circular, oval, D-shaped and other similar shapes. In some examples the outer boundary shape of a coil may conform to the shape of the device housing, e.g., to the shape of base 104 depicted in FIG. 3.

The positions of first lead 304 and second lead 306 are not limited to any particular arrangement, such as the arrangement as shown in FIG. 5A. In some examples leads 304 and 306 may extend from other positions of the coil winding of the receive coil 300, including having first lead 304 and second lead 306 extend from different portions of the coil windings so that these leads do not extend from portions of the receive coil that are in close proximity to one another.

Electrical conductor 302 is not limited to being formed from any particular type of material, and may be formed from any type of electrical conductor, including a conductive metal, such as copper, that is formed into a wire and may be easily bent to form the desired shape of the coil winding used to form receive coil 70. The electrical conductor used to form receive coil 300 in FIG. 5A in some examples may include an insulative material, such as enamel, coated over the exterior surface of the conductor to provide an insulative layer between the individual coil windings. In various examples, the electrical conductor used to form receive coil 300 is a multi-strand conductor, such as Litz wire, wherein the electrical conductor used to form each winding is insulated along the outer surface of the electrical conductor, for example using a coating, such as enamel, to reduce the skin effect of the electrical conductor.

In some examples, the receive coil 300 as illustrated in FIG. 5A may be manipulated to include a single half-twist of one portion of the receive coil 300 so that the receive coil forms the shape of an infinity-loop as illustrated in FIG. 5A. As shown in FIG. 5A, the windings of electrical conductor 302 form a first loop 308, and a second loop 310 coupled to the first loop at crossover area 312. A winding of receive coil 300 having an end coupled to first lead 304 extends from first lead 304 and around the outer-most winding of first loop 308, and then to crossover area 312. This same winding extends from crossover area 312 to form a portion of the winding included in second loop 310 before again returning to the crossover area 312. Windings of receive coil 300 continue to form a progressive series of windings forming a portion of the winding in first loop 308, extending to the crossover area 312, and forming a winding in the second loop 310 before again returning to the crossover area 312, until an end of conductor 302 is reached that is coupled to second lead 306. The total number of turns formed by the windings passing around the first loop 308 through the crossover area 312 and around the second loop 310 is not limited to any particular number of turns, and is some examples may be ten turns or some other number of turns.

In examples where the infinity-loop shape of receive coil 300 was first formed in the shape of a circular or oval winding as shown in FIG. 5A, all of the electrical conductor 302 aligned in the crossover area 312 may be either above or below all of the other portions of the electrical conductor 302 that are aligned with one another and pass through the crossover area. For example, all portions of the electrical conductor 302 that align with one another when entering and exiting the crossover area 312 are all either above (e.g., pass on top of as shown in FIG. 5A) or are all below (e.g., pass underneath) other conductors. Thus in some examples, the thickness dimension of the infinity shaped coil at the crossover area 312 may be greater than the thickness dimension of two or more portions of the electrical conductor 302 combined.

As an alternative to first forming receive coil 300 as a single loop and then twisting a portion of the loop used to form the infinity shaped coil as illustrated in FIG. 5A, the infinity shaped coil of FIG. 5A may be wound initially in the figure-eight pattern to form the infinity shaped coil. In various examples of winding the figure-eight pattern to form the infinity shaped coil, the winding in the outermost winding of electrical conductor 302 around first loop 308 may be arranged as the inner-most winding of the electrical conductor 302 around second loop 310. The routing of electrical conductor 302 may continue in a manner such that the second outermost portion of electrical conductor 302 within first loop 308 continues as the second-most inner portion of the electrical conductor 302 formed within the second loop 310. By continuing to alternatively form a winding of receive coil 300 using this outermost versus innermost pattern relative to first loop 308 and second loop 310, the thickness of the windings at the crossover area 312 may be maintained to no more than a thickness dimension of two of the windings of electrical conductor 302 combined. This pattern may therefor provide a flatter or less thick coil winding in the portion of the electrical conductor 302 that crossover one another within the crossover area 312.

Regardless of whether receive coil 300 was formed into the infinity shaped coil by twisting a circular or oval shaped coil or by winding the receive coil in a figure-eight pattern, receive coil 300 may be formed into a curved shape in some examples. When formed into a curved shape, as shown in FIG. 5A, receive coil 300 may or may not be affixed to a ferrite sheet (not shown in FIG. 5A), and receive coil 300 positioned so that curvature of receive coil 300 corresponds to the inner surface for example of an antenna window or along another portion of the housing of a power receiving device, such as implantable medical device 14 or IMD 100 described above in relation to FIGS. 1 and 3.

In the example of FIG. 5A, receive coil 300 is bent along the length of longitudinal axis 314 so that the longitudinal dimension corresponding to the longitudinal axis 314 of the receive coil forms a curved shape 316. In other examples, receive coil 300 may form a flat, planar coil, which may conform to the shape of a flat portion of the device housing, such as cover 101 described above in relation to FIG. 3.

The amount of curvature along longitudinal axis 314 may correspond to the curvature of the inner surface of the housing of IMD 100, e.g., of base 104, so that receive coil 300 may be affixed along and positioned directly adjacent to a portion of the inner of the housing. In some examples, receive coil 300 is affixed to a ferrite sheet. The shape of receive coil 300, e.g., the amount of curvature of the receive coil 300 may be formed so that receive coil 300 may be affixed to a surface of the ferrite sheet, and the surface of the ferrite sheet opposite the surface where the receive coil is attached may be affixed in contact with and directly proximity to a portion of the inner surface the device housing.

In other examples, receive coil 300 is not affixed to a ferrite sheet. Receive coil 300 may be bent along the length of longitudinal axis 314, and affixed in direct contact with and directly adjacent to the inner surface of the housing.

FIG. 5B illustrates an example of electrical conductors configured to form receive coil 350 for an implantable medical device, or some other wireless power receiving device according to various examples described in this disclosure. In the example of FIG. 5B, a first electrical conductor is formed into a first coil winding indicated by bracket 352, the first electrical conductor having a first end 356 at one end of the coil winding, and a second end 358 at the end of the electrical conductor opposite first end 356. First coil winding may be made of any type of electrical conductor, including the conductive wire such as Litz wire as described throughout this disclosure. In other examples, the dual receive coil 350 in the example of FIG. 5B may be arranged as a triple, quadruple or any other number of coil windings (not shown in FIG. 5B).

The example of FIG. 5B also depicts a second electrical conductor formed into a second coil winding indicated by bracket 354, the second electrical conductor having a first end 360 at one end of the coil winding, and a second end 362 at the end of the electrical conductor opposite second end 356. The second coil winding may also be made of any type of electrical conductor. The type of material used, the general dimensions, and the number of turns used to form the second coil winding are the same or similar to those used to form the first coil winding.

The first coil winding and the second coil windings may be affixed to a ferrite sheet, or to separate ferrite sheets, where the ferrite sheets may then be affixed to an inner surface of an interior cavity of wireless power receiving device, such as IMD 100 of FIG. 3. In some examples, the inner surface of the interior cavity of the device may form a curved surface, wherein the first coil winding and the second coil winding may be positioned next to one another so that a longitudinal axis extending through each of the first coil winding and the second coil winding extends around or along a perimeter of the inner surface and longitudinal axis 364 may conform the curvature (shown by double-headed arrow 366) of the inner surface of the implantable medical device). The curvature separates the two loops of the dual-winding coil configuration into separate planes, and thus allows the dual-winding coil configuration to generate an induced current flow when a magnetic field is imposed onto one or both of the coil windings.

Similarly, the first coil winding and the second coil winding may be placed on two separate surfaces of the device housing when the two surfaces do not define a curved surface. For example, the plane of the first coil winding may be located at some angle with respect to the plane of the second coil winding, where in some examples the angle may be defined by the geometry of the device. In other words, the angle may be based on a shape of the housing, e.g., in which two surfaces are at an angle. In other examples the angle may be defined by a surface of the housing and a surface of some other portion of the wireless power receiving device, such as of electrical energy storage device 106, circuitry 120 or some other portion of the device, as described above in relation to FIG. 3.

The second end 358 of the first coil winding is electrically coupled to the second end 362 of the second coil winding. The connection coupling the second end 358 and the second end 362 in some examples may be formed on a circuit board or a hybrid substrate (not shown in FIG. 5B), thus allowing each of the first coil winding and the second coil winding to be coupled together either before or after the coils have been affixed in place within the housing of the implantable medical device. In the example of FIG. 5B, second end 358 of the first coil winding extends to form the outermost winding of the first coil winding, and the innermost winding of the second coil winding extends to second end 362, which is directly coupled to second end 358. The first end 356 of the first coil winding and the first end 360 of the second coil winding are configured to be coupled to power receiving circuitry, such as recharging circuitry 30 as illustrated and described with respect to FIG. 2.

The first coil winding and the second coil winding as illustrated in FIG. 5B may be referred to as a dual-winding coil configuration forming a two-loop coil winding. The dual-winding coil configuration illustrated and described with respect to receive coil 350 may be included in place of the infinity shaped coil(s) in any of the receive antenna configurations described throughout this disclosure. For examples, the dual-winding coil configuration as shown in FIG. 5B may be substituted for the infinity shaped receive coil 300 illustrated and described with respect to FIG. 5A. In examples in which the two loops of the dual-winding coil configuration of receive coil 350 are positioned in different planes relative to one another, the dual-winding coil configuration may provide a recharging current induced into one or both of the coil winding when a magnetic field is imposed onto the dual-winding coil configuration from a variety of different magnetic field direction relative to the orientation of the dual-winding coil configuration.

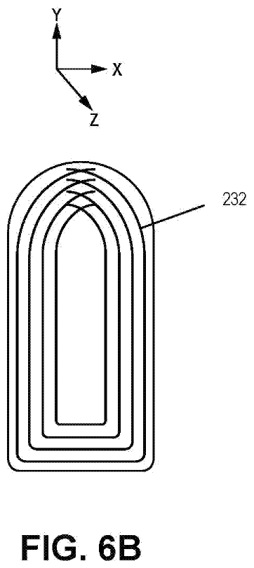

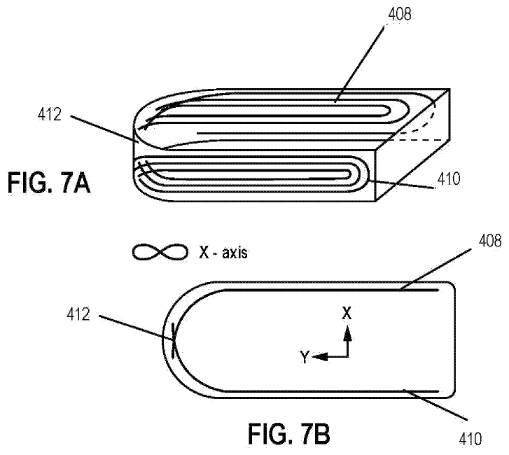

FIG. 6A is a conceptual diagram of a flat, spiral shaped Z-coil, according to one or more techniques of this disclosure. Z-coil 228 is an example of Z-coil 118 described above in relation to FIG. 3 and may be located parallel to a cover of the housing for the IMD, e.g., cover 101. In some examples, Z-coil 228 may also have a flexible ferrite sheet 230 proximal to Z-coil 228. As with Z-coil 218 of FIG. 4B, Z-coil 228 may be oval, circular, rectangular or any other shape, e.g., a shape that conforms to the housing of the wireless power receiving device. In some examples, a shape that conforms to the size of the housing, may provide an advantage of a larger aperture when compared to a shape smaller than the housing.

FIG. 6B is a conceptual diagram of a flat, folded infinity wound coil configured as a Z-coil according to one or more techniques of this disclosure. Z-coil 232, in the example of FIG. 6B may be arranged as folded infinity coil, e.g., similar to receive coil 300, or a multiple coil receive coil, e.g., receive coil 350 as described above in relation to FIGS. 5A and 5B.

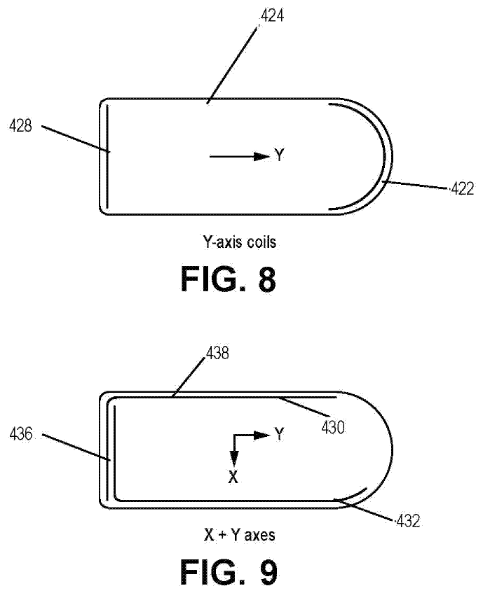

FIGS. 7A and 7B are conceptual diagrams illustrating an example spiral-wound coil implemented as an X-coil, according to one or more techniques of this disclosure. The isometric view of FIG. 7A shows an example receive coil 400 implemented as an infinity coil similar to receive coil 300 of FIG. 5A. Receive coil 400 includes first coil winding 408, second coil winding 410 and cross over point 412. FIG. 7B shows receive coil 400 in a top view. In other examples, receive coil 400 may also be implemented as a dual coil, similar to coil 350 as described above in relation to FIG. 5B.

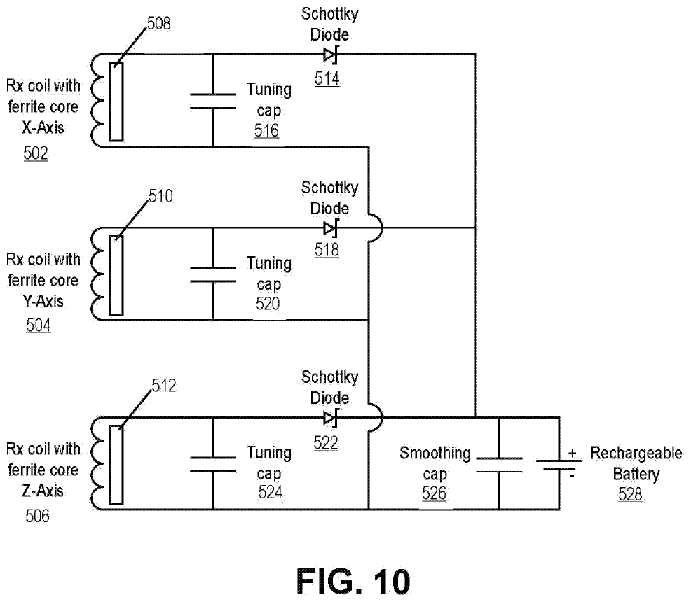

FIG. 8 is a conceptual diagram illustrating an example, of receive coils implemented as a Y-coil according to one or more techniques of this disclosure. In the example of FIG. 8, receive coil 428 may be implemented as a planar coil and aligned with a surface of housing 424 of the wireless power receiving device. Similarly, coil 422 may be aligned with a different surface of housing 424, which in the example of FIG. 8 may be a curved portion of the housing. The apertures for coils 422 and 428 may be aligned in the Y-direction, as shown. In some examples, either or both of coils 422 and 428 may be proximal to a ferrite sheet. In some examples, either or both of coils 422 and 428 may be a spiral wound, or some other coil arrangement, e.g., similar to receive coils 300 and 350 of FIGS. 5A and 5B.

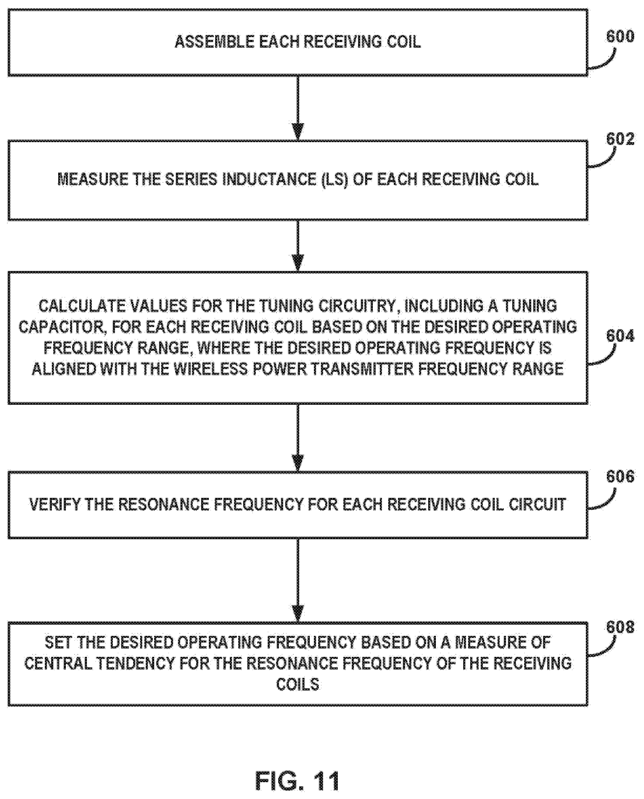

FIG. 9 is a conceptual diagram illustrating an example receive coil implemented to receive wireless energy in multiple planes, according to one or more techniques of this disclosure. In the example of FIG. 9, receive coil 430 may include a first portion 438 aligned with an aperture with the X-axis and a second portion 436 with the aperture aligned with the Y-axis. In other examples, receive coils 430 and 432 may include arrangements to align with any of the X, Y or Z axes. In some examples, receive coils 430 and 432 may be implemented as infinity wound coils, such as coil 300, or a multi-winding coils, such as coil 350 shown in FIGS. 5A and 5B. For example, coil 430 may be a dual winding receive coil with a first coil winding as first portion 438 and the second coil winding as second portion 436.

In other words, as described above in relation to FIGS. 4A, 4B, 6B, 7A, 7B, 8 and 9, the IMD of this disclosure may have multiple coils serving each axis in any combination of arrangements described above. For example, the Z-axis could be wound on the ferrite core along with X- and Y-axis in addition to a separately placed coil along the periphery of the housing (not shown in FIG. 9).

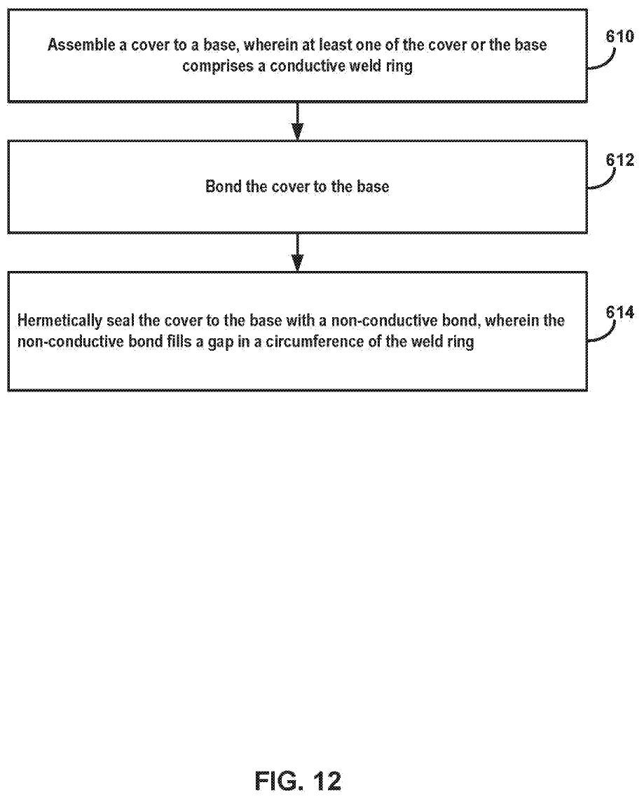

FIG. 10 is a schematic diagram illustrating an example three coil wireless power receiving circuit, according to one or more techniques of this disclosure. The example of circuit 500 includes three receive coils, but in other examples, the power receiving circuit may include more or fewer receive coils. Each receive coil, Rx coil 502, Rx coil 504 and Rx coil 506 are connected in parallel with smoothing capacitor 526 and an electrical energy storage device, which is rechargeable battery 528 in the example of FIG. 10. In other examples, the electrical energy storage device may be a capacitor or similar storage device, as described above in relation to FIG. 3 for electrical energy storage device 106.

Rx coil 502 is configured as the X-axis coil and is may be located near ferrite core 508 or wound onto ferrite core 508. Tuning capacitor 516 connects in parallel to Rx coil 502. One terminal of Rx coil 502 connects to the positive terminal of battery 528 through Schottky diode 514. Similarly, Rx coil 504, is configured as the Y-axis coil and is located near ferrite or wound onto core 510. Tuning capacitor 520 connects in parallel to Rx coil 504. One terminal of Rx coil 504 connects to the positive terminal of battery 528 through Schottky diode 518. Also, Rx coil 506, is configured as the Z-axis coil and is located near or wound onto ferrite core 512. Tuning capacitor 524 connects in parallel to Rx coil 506. One terminal of Rx coil 506 connects to the positive terminal of battery 528 through Schottky diode 522. In some examples, any of the Rx coils may be assembled with, or without, the ferrite. In some examples, the ferrite is a ferrite core, while in other examples the ferrite is a ferrite sheet, as described above in relation to FIGS. 3, 4, 6, 7A and 7B.

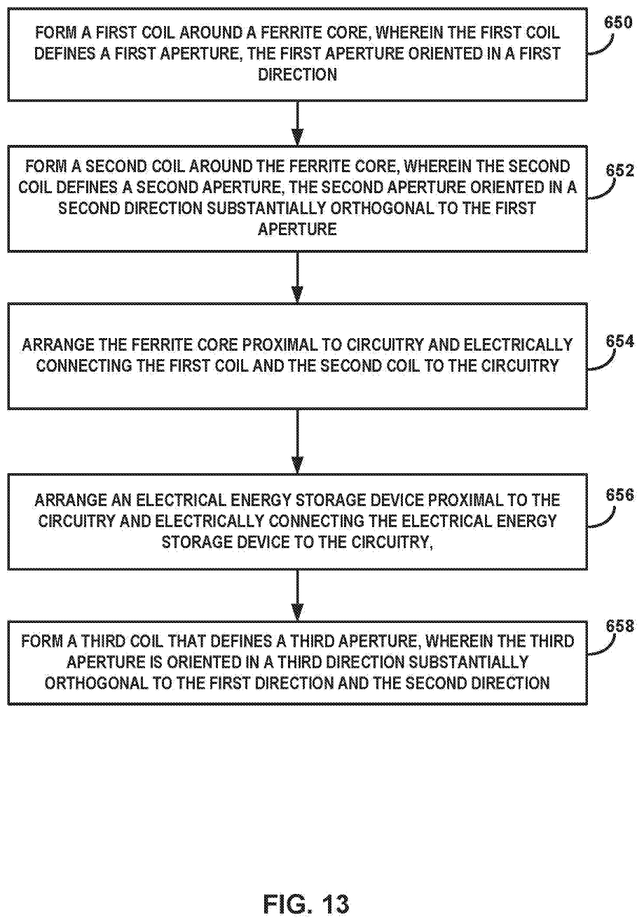

FIG. 11 is a flow chart illustrating an example method of manufacturing an implantable medical device according to one or more techniques of this disclosure. After assembling each receiving coil (600), the production facility may measure the series inductance (Ls) of each receiving coil (602). As described above in relation to FIG. 3, because the size, shape and number of windings for each receive coil, may be different, the measured inductance, Ls, for each coil may differ.

In some examples, the production facility may calculate values for the components to be used in the tuning circuitry for each coil, such as a tuning capacitor, as described above in relation to FIG. 10 (604). In some examples, the selected tuning capacitor, and other components may be matched for each receive coil based on the measured value for Ls, as well as the desired operating frequency range for the device. The tuning circuitry may align the resonant frequency of each of the coils to each other. The operating frequency range may align with an operating frequency range output from wireless power transmitter, e.g., external computing device 22 and primary coil 20. In this disclosure, to “align” the resonant frequency may describe tuning the resonant frequency of the coil plus tuning circuitry such that each resonant frequency of each respective coil is within a desired operating frequency range, though not necessarily perfectly matched to each other.

In some examples, the production facility may verify the resonance frequency for each receiving coil circuit, e.g., after assembling the receive coil, tuning capacitors, diodes and other circuitry (606). In some examples, the desired operating frequency for the device may be set based on an average, median, mode or some other measure of central tendency for the group of receiving coils (608). The desired operating frequency may be within an operating frequency range that aligns with the operating frequency range of the power transmitting devices.

FIG. 12 is a flow chart illustrating an example method of manufacturing an implantable medical device according to one or more techniques of this disclosure. As described above in relation to FIG. 3, the housing for the wireless power receiving device of this disclosure may hermetically seal the receiving coils and other components of the device. In some examples, the housing may include a base, e.g., base 104 and one or more covers, e.g., cover 101 and/or cover 128.

A production facility may assemble a cover to a base, e.g., to form a housing assembly (610). Bonding equipment may bond the cover to the base (612). In some examples each of base 104 and cover 101 (or 128) may include a weld ring around the circumference of the mating surface between base 104 and cover 101. The weld ring may comprise a conductive material. The bonding equipment may include laser welding, low temperature bonding, e.g., a sputter process, or some other bond which acts to seal the cover to the base. The completed weld ring, after bonding, may act as an electrode to sense bioelectrical signals and deliver electrical stimulation to target tissue of the patient.

In some examples the weld ring, on both cover 101 and base 104, may include a non-conductive gap, e.g., gap 124, which may prevent eddy currents from completing path around the circumference of the weld ring. To finalize the hermetic seal, a non-conductive bond may seal cover 101 to base 104 across gap 124.

FIG. 13 is a flow chart illustrating an example method of manufacturing a wireless power receiving device according to one or more techniques of this disclosure. As shown by FIGS. 3 and 4, in some examples, the wireless power receiving device, e.g., IMD 100, may include an X-coil 212 and a Y-coil 214 wrapped around a ferrite core 226, as well as a Z-coil 218, which in the example of FIG. 3, may conform to the shape of the housing, e.g., to the shape of base 104.

In some examples, to build the assembly may include first forming a first coil around ferrite core 226, in which the first coil defines a first aperture, the first aperture oriented in a X-direction (650). Next, form a second coil around ferrite core 226, where the second coil defines a second aperture, the second aperture oriented in the Y-direction and substantially orthogonal to the first aperture for X-coil 212 (652).

As shown in FIG. 3, arrange the ferrite core and coils assembly proximal to circuitry 120 and electrically connecting X-coil 112 and the Y-coil 114 to circuitry 120 (654). Either before or after connecting the coils to circuitry 120, arrange electrical energy storage device 106 proximal to circuitry 120 and electrically connecting electrical energy storage device 106 to circuitry 120 (656).

For the Z-coil, form a third coil that defines a third aperture, wherein the third aperture is oriented in a third direction substantially orthogonal to the X-direction and the Y-direction. In the example of FIGS. 3, 4A and 4B, the aperture size for the Z-coil is larger than for either of the X-coil and the Y-coil. In some examples, the aperture size for the Z-coil may larger than the X-coil aperture and the Y-coil aperture. In some examples the Z-coil aperture may be 1.5 times larger, twice as large, five times, ten times or some other size by comparison to either of the X-coil or the Y-coil aperture.

The techniques of this disclosure may also be described in the following examples.

Example 1: An implantable medical device comprising circuitry configured to receive wireless power; an electrical energy storage device configured to provide electrical energy to the circuitry, wherein the circuitry is configured to charge the energy storage device using the wireless power; a secondary antenna configured to configured to receive the wireless power and conduct the wireless power to the circuitry, the secondary antenna comprising a first coil that defines a first aperture, the first aperture oriented in a first direction; a second coil that defines a second aperture, the second aperture oriented in a second direction substantially orthogonal to the first aperture; a third coil that defines a third aperture, wherein the third aperture: is oriented in a third direction substantially orthogonal to the first direction and the second direction; has at least twice the area as either of the first aperture and the second aperture.

Example 2: The device of example 1, further comprising a non-conductive housing that encloses and hermetically seals the circuitry, the electrical energy storage device and the secondary antenna inside the housing, wherein the third coil is located along a periphery of the housing.

Example 3: The device of example 2, wherein the third coil surrounds the circuitry, the electrical energy storage device, the first coil and the second coil.

Example 4: The device of any of examples 2 and 3, further comprising a flexible ferrite located along the periphery of the housing; and conforming to a shape of the third coil.

Example 5: The device of any of examples 1 through 4, further comprising a ferrite core, wherein the first coil and the second coil are wrapped around the ferrite core.

Example 6: The device of any of examples 1 through 5, wherein each of the first coil, the second coil and the third coil of the secondary antenna simultaneously conduct the wireless power to the circuitry.

Example 7: The device of any of examples 1 through 6, wherein the circuitry comprises tuning circuitry for the first coil, wherein the tuning circuitry comprises a tuning capacitor; wherein the tuning circuitry is configured to align a first resonant frequency of the first coil to a second resonant frequency of the second coil.

Example 8: A wireless power transfer system comprising two or more electrodes configured to be placed proximal to target tissue of a patient; an implantable medical device comprising circuitry configured to: measure bioelectrical signals of the patient via the two or more electrodes; and receive wireless power; an electrical energy storage device configured to provide electrical energy to the circuitry, wherein the circuitry is configured to charge the energy storage device using the wireless power; a secondary antenna configured to configured to receive the wireless power and conduct the wireless power to the circuitry, the secondary antenna comprising a first coil that defines a first aperture, the first aperture oriented in a first direction; a second coil that defines a second aperture, the second aperture oriented in a second direction substantially orthogonal to the first aperture; a third coil that defines a third aperture, wherein the third aperture: is oriented in a third direction substantially orthogonal to the first direction and the second direction; has at least twice the area as either of the first aperture and the second aperture.

Example 9: The system of example 8, wherein the implantable medical device is further configured to deliver electrical stimulation therapy to the patient via the two or more electrodes.

Example 10: The system of any of examples 8 and 9, further comprising a non-conductive housing that encloses and hermetically seals the circuitry, the electrical energy storage system and the secondary antenna inside the housing, wherein the third coil is located along the periphery of the housing.

Example 11: The system of example 10, wherein the third coil surrounds the circuitry, the electrical energy storage system, the first coil and the second coil.

Example 12: The system of any of examples 10 and 11, further comprising a flexible ferrite located along the periphery of the housing; and conforming to a shape of the third coil.

Example 13: The system of any of examples 8 through 12, further comprising a ferrite core, wherein the first coil and the second coil are wrapped around the ferrite core.

Example 14: The system of any of examples 8 through 13, wherein each of the first coil, the second coil and the third coil of the secondary antenna simultaneously conduct the wireless power to the circuitry.

Example 15: The system of any of examples 8 through 14, wherein the circuitry comprises tuning circuitry for the first coil, wherein the tuning circuitry comprises a tuning capacitor; wherein the tuning circuitry is configured to align a first resonant frequency of the first coil to a second resonant frequency of the second coil.

Example 16: The system of any of examples 8 through 15, further comprising a wireless power transmitting device configured to output and control wireless power delivery to the implantable medical device.

Example 17: A method of manufacturing a wireless power receiving device comprising forming a first coil around a ferrite core, wherein the first coil defines a first aperture, the first aperture oriented in a first direction; forming a second coil around the ferrite core, wherein the second coil defines a second aperture, the second aperture oriented in a second direction substantially orthogonal to the first aperture; arranging the ferrite core proximal to circuitry and electrically connecting the first coil and the second coil to the circuitry; arranging an electrical energy storage device proximal to the circuitry and electrically connecting the electrical energy storage device to the circuitry, wherein the circuitry is configured to receive wireless power via the first coil and the second coil, wherein the circuitry is configured to charge the energy storage device using the wireless power received during a charging session, wherein the electrical energy storage device is configured to provide electrical energy to the circuitry; and forming a third coil that defines a third aperture, wherein the third aperture: is oriented in a third direction substantially orthogonal to the first direction and the second direction; has at least twice the area as either of the first aperture and the second aperture.

Example 18: The method of example 17, further comprising arranging the circuitry, the electrical energy storage device, the first coil, the second coil and the third coil in a non-conductive housing configured to enclose and hermetically seal the circuitry, the electrical energy storage device, the first coil, the second coil and the third coil inside the housing, wherein the third coil is located along a periphery of the housing.

Example 19: The method of example 18, wherein the housing further comprises two or more electrodes configured to be placed proximal to target tissue of a patient, the method further comprising, connecting the circuitry to the two or more electrodes.

Example 20: The method of any of examples 18 and 19, further comprising, installing a flexible ferrite located along the periphery of the housing, wherein the flexible ferrite conforms to a shape of the third coil.

Example 21: A method of manufacturing a wireless power receiving device comprising assembling a receiving coil of a plurality of receiving coils, wherein each receiving coil of the plurality of receiving coils comprises one or more coil windings comprising a conductive material configured to carry electrical current; measuring the inductance of each receiving coil; calculating values for respective tuning circuitry associated with each receiving coil based on an operating frequency range for the respective receiving coil; and verifying a resonance frequency for each receiving coil circuit, wherein each receiving coil circuit comprises the respective receiving coil and respective tuning circuitry.

Example 22: An implantable medical device comprising two or more electrodes configured to be placed proximal to target tissue of a patient; circuitry; and a non-conductive, hermetically sealed housing: configured to enclose the circuitry, wherein the circuitry is configured to measure bioelectrical signals of the patient via the two or more electrodes; comprising a conductive ferrule configured to: hermetically seal the housing; and act as a first electrode of the two or more electrodes.

Example 23: The device of example 22, wherein the circuitry is configured to receive radio frequency (RF) energy through the housing.

Example 24: The device of example 23, wherein the conductive ferrule includes a non-conductive break configured to avoid eddy currents in the conductive ferrule.

Example 25: The device of any of examples 22 through 24, wherein the housing comprises a cover, wherein the conductive ferrule is configured to hermetically seal the cover of the housing; wherein a second electrode is located on the cover, such that: the cover and second electrode form a hermetic seal with the cover, the second electrode is electrically isolated on the cover from the conductive ferrule; the second electrode connects to the circuitry through the cover.

Example 26: The device of example 25, wherein the cover is a first cover, the housing of the device further comprising a second cover, wherein: the second cover is located on an opposite side of the housing from the first cover; and the second cover is non-conductive.

Example 27: The device of any of 22 through 26, wherein the cover comprises a sapphire material.

Example 28: The device of any of examples 1 through 27, wherein the conductive ferrule uses a temperature diffusion bond to hermetically seal the housing.

Example 29: The device of any of examples 1 through 28, wherein the circuitry is further configured to deliver electrical stimulation therapy to the patient via the two or more electrodes.

Example 30: The device of example 29, wherein the circuitry is configured to deliver the electrical stimulation therapy based on one or more of:
the measured bioelectrical signals;
information from one or more sensors operatively coupled to the circuitry, or
a message received via communication circuitry operatively coupled to the circuitry.

Example 31: A wireless power transfer system comprising one or more antennae configured to receive wireless power from a power transmitting device; an implantable medical device (IMD) comprising two or more electrodes configured to be placed proximal to target tissue of a patient; circuitry; and a non-conductive, hermetically sealed housing: configured to enclose the circuitry, wherein the circuitry is configured to measure bioelectrical signals of the patient via the two or more electrodes; comprising a conductive ferrule configured to: hermetically seal the housing; and act as a first electrode of the two or more electrodes.

Example 32: The system of example 31, wherein the circuitry is configured to receive radio frequency (RF) energy through the housing via the one or more antennae.

Example 33: The system of example 31 and 31, wherein the conductive ferrule includes a non-conductive break configured to avoid eddy currents in the conductive ferrule.

Example 34: The system of any of examples 31 through 32, wherein the housing comprises a cover, wherein the conductive ferrule is configured to hermetically seal the cover of the housing; wherein a second electrode is located on the cover, such that: the cover and second electrode form a hermetic seal with the cover, the second electrode is electrically isolated on the cover from the conductive ferrule; the second electrode connects to the circuitry through the cover.

Example 35: The system of example 33, wherein the cover is a first cover, the housing of the IMD further comprising a second cover, wherein: the second cover is located on an opposite side of the housing from the first cover; and the second cover is non-conductive.

Example 36: The system of example 33, wherein the cover comprises a sapphire material.

Example 37: The system of any of examples 31 through 35, wherein the conductive ferrule uses a temperature diffusion bond to hermetically seal the housing.

Example 38: The system of any of examples 31 through 36, wherein the circuitry is further configured to deliver electrical stimulation therapy to the patient via the two or more electrodes.

Example 39: A method of manufacturing a wireless power receiving device comprising assembling a cover to a base, wherein: a first circumference of the cover aligns with second circumference of the base; the circumference of the cover comprises a conductive weld ring; the circumference of the cover comprises a non-conductive gap in the weld ring; bonding the cover to the base; and hermetically sealing the cover to the base, where hermetically sealing the cover to the base comprises: bonding the non-conductive gap in the cover to the base with a non-conductive bond.

In one or more examples, the functions described above may be implemented in hardware, software, firmware, or any combination thereof. For example, the various components of FIGS. 1, 2 and 3, such as external computing device 22, processing circuitry 34 and circuitry 12 may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

The term “non-transitory” may indicate that the storage medium is not embodied in a carrier wave or a propagated signal. In certain examples, a non-transitory storage medium may store data that can, over time, change (e.g., in RAM or cache). By way of example, and not limitation, such computer-readable storage media, may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Combinations of the above should also be included within the scope of computer-readable media.

Instructions may be executed by one or more processors, such as one or more DSPs, general purpose microprocessors, ASICs, FPGAs, or other equivalent integrated or discrete logic circuitry. Accordingly, the term “processor” and “processing circuitry,” as used herein, such as processing circuitry 34, may refer to any of the foregoing structure or any other structure suitable for implementation of the techniques described herein. Also, the techniques could be fully implemented in one or more circuits or logic elements.

The techniques of this disclosure may be implemented in a wide variety of devices or apparatuses, including, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples of the disclosure have been described. These and other examples are within the scope of the following claims.

1. An implantable medical device, the device comprising:
circuitry configured to receive wireless power;
an electrical energy storage device configured to provide electrical energy to the circuitry, wherein the circuitry is configured to charge the energy storage device using the wireless power; and
a secondary antenna configured to receive the wireless power and conduct the wireless power to the circuitry, the secondary antenna comprising:
a first coil that defines a first aperture, the first aperture oriented in a first direction;
a second coil that defines a second aperture, the second aperture oriented in a second direction substantially orthogonal to the first aperture; and
a third coil that defines a third aperture, wherein the third aperture:
is oriented in a third direction substantially orthogonal to the first direction and the second direction; and
has at least twice an area of either of the first aperture and the second aperture.

2. The device of claim 1, further comprising a non-conductive housing that encloses and hermetically seals the circuitry, the electrical energy storage device and the secondary antenna inside the housing, wherein the third coil is located along a periphery of the housing.

3. The device of claim 1, wherein the third coil surrounds the circuitry, the electrical energy storage device, the first coil, and the second coil.

4. The device of claim 2, further comprising a flexible ferrite,
located along the periphery of the housing; and
conforming to a shape of the third coil.

5. The device of claim 1, further comprising a ferrite core, wherein the first coil and the second coil are wrapped around the ferrite core.

6. The device of claim 1, wherein each of the first coil, the second coil and the third coil of the secondary antenna simultaneously conduct the wireless power to the circuitry.

7. The device of claim 1,
wherein the circuitry comprises tuning circuitry for the first coil,
wherein the tuning circuitry comprises a tuning capacitor; and
wherein the tuning circuitry is configured to align a first resonant frequency of the first coil to a second resonant frequency of the second coil.

8. A wireless power transfer system, the system comprising:
two or more electrodes configured to be placed proximal to target tissue of a patient;
an implantable medical device comprising:
circuitry configured to:
measure bioelectrical signals of the patient via the two or more electrodes; and
receive wireless power;
an electrical energy storage device configured to provide electrical energy to the circuitry, wherein the circuitry is configured to charge the energy storage device using the wireless power; and
a secondary antenna configured to configured to receive the wireless power and conduct the wireless power to the circuitry, the secondary antenna comprising:
a first coil that defines a first aperture, the first aperture oriented in a first direction;
a second coil that defines a second aperture, the second aperture oriented in a second direction substantially orthogonal to the first aperture; and
a third coil that defines a third aperture, wherein the third aperture:
is oriented in a third direction substantially orthogonal to the first direction and the second direction; and
has at least twice the area as either of the first aperture and the second aperture.

9. The system of claim 8, wherein the implantable medical device is further configured to deliver electrical stimulation therapy to the patient via the two or more electrodes.

10. The system of claim 8, further comprising a non-conductive housing that encloses and hermetically seals the circuitry, the electrical energy storage system and the secondary antenna inside the housing, wherein the third coil is located along a periphery of the housing.

11. The system of claim 8, wherein the third coil surrounds the circuitry, the electrical energy storage system, the first coil and the second coil.

12. The system of claim 10, further comprising a flexible ferrite:
located along the periphery of the housing; and
conforming to a shape of the third coil.

13. The system of claim 8, further comprising a ferrite core, wherein the first coil and the second coil are wrapped around the ferrite core.

14. The system of claim 8, wherein each of the first coil, the second coil and the third coil of the secondary antenna simultaneously conduct the wireless power to the circuitry.

15. The system of claim 8,
wherein the circuitry comprises tuning circuitry for the first coil;
wherein the tuning circuitry comprises a tuning capacitor; and
wherein the tuning circuitry is configured to align a first resonant frequency of the first coil to a second resonant frequency of the second coil.

16. The system of claim 8, further comprising a wireless power transmitting device configured to output and control wireless power delivery to the implantable medical device.

17. A method of manufacturing a wireless power receiving device comprising:
forming a first coil around a ferrite core, wherein the first coil defines a first aperture, the first aperture oriented in a first direction;
forming a second coil around the ferrite core, wherein the second coil defines a second aperture, the second aperture oriented in a second direction substantially orthogonal to the first aperture;
arranging the ferrite core proximal to circuitry and electrically connecting the first coil and the second coil to the circuitry;
arranging an electrical energy storage device proximal to the circuitry and electrically connecting the electrical energy storage device to the circuitry, wherein the circuitry is configured to receive wireless power via the first coil and the second coil, wherein the circuitry is configured to charge the energy storage device using the wireless power received during a charging session, wherein the electrical energy storage device is configured to provide electrical energy to the circuitry; and
forming a third coil that defines a third aperture, wherein the third aperture:
is oriented in a third direction substantially orthogonal to the first direction and the second direction; and
has at least twice the area as either of the first aperture and the second aperture.

18. The method of claim 17, further comprising:
arranging the circuitry, the electrical energy storage device, the first coil, the second coil and the third coil in a non-conductive housing configured to enclose and hermetically seal the circuitry, the electrical energy storage device, the first coil, the second coil and the third coil inside the housing, wherein the third coil is located along a periphery of the housing.

19. The method of claim 18, wherein the housing further comprises two or more electrodes configured to be placed proximal to target tissue of a patient, the method further comprising, connecting the circuitry to the two or more electrodes.

20. The method of claim 18, further comprising, installing a flexible ferrite located along the periphery of the housing, wherein the flexible ferrite conforms to a shape of the third coil.
